(19) United States
(12) Patent Application Publication
(10) Pub. No.: US 2025/0268721 A1
(43) Pub. Date: Aug. 28, 2025

(54) LOCKING MECHANISM FOR JOINING BONES AND RELATED METHOD

(71) Applicant: BEACON BIOMEDICAL, LLC, Jupiter, FL (US)

(72) Inventors: Marc J. Levine, Richboro, PA (US); Jonathan M. Lewis, Palm Beach Gardens, FL (US)

(21) Appl. No.: 19/039,617

(22) Filed: Jan. 28, 2025

Related U.S. Application Data
(60) Provisional application No. 63/626,106, filed on Jan. 29, 2024.

(51) Int. Cl. A61F 2/44; A61F 2/30

(52) U.S. Cl. CPC A61F 2/4425; A61F 2/30771; A61F 2/447; A61F 2002/30477; A61F 2002/30772; A61F 2002/30845

(57) ABSTRACT

An ax fastener or locking mechanism for an implant or interbody spacer for promoting fusion between first and second bone portions or vertebral bodies includes a peripheral wall defining an internal space, a web/lattice within the internal space and a rotatable fastener connected to the peripheral wall. The rotatable fastener includes a shaft, a first blade and a second blade. The rotatable fastener is movable from an insertion configuration to an expanded configuration. The first and second blades are positioned between upper and lower planes defined by the peripheral wall in the insertion configuration and extend beyond the upper and lower planes in the expanded configuration.

### PRIORITY CLAIM

In accordance with 37 C.F.R. 1.76, a claim of priority is included in an Application Data Sheet filed concurrently herewith. Accordingly, the present invention claims priority based upon U.S. Provisional Patent Application No. 63/626,106 filed Jan. 29, 2024 entitled “LOCKING MECHANISM FOR JOINING BONES AND RELATED METHOD” the contents of which are incorporated herein by reference.

### BACKGROUND OF THE INVENTION

Spinal fusions including cervical, thoracic, lumbar and sacral often utilize an implant (index implant) or interbody spacer that is positioned between vertebral bodies and typically facilitates fusion of the two vertebral bodies. These implants or interbody spacers have been made of various materials including autograft, allograft, stainless steel, titanium, ceramics, polymerics, as well as other materials. The implants or interbody spacers have been manufactured in a multitude of ways including legacy machine work as well as 3D printing with open interior structures, in addition to truss technology and arch technology. Surfaces have been consistent with the remainder of the implant as well as surface coatings with roughened surfaces added to enhance bone incorporation and fusion.

Historically, the interbody device or implant can be held in place by “press fitting.” These press-fit implants can be further stabilized by adding plates that may bridge over the implant between the two vertebral bodies and further control the motion segment or “block” the implant from coming out or backing out. Screws and rods have also been used in a similar fashion to stabilize the motion segment which may include posterior placement via the pedicles/lateral masses of the vertebral bodies. More recently, “stand alone” interbody devices have been designed.

There are interbody devices that also are considered “stand alone” since they provide fixation devices that typically go through the interbody and penetrate the endplate(s) of the bone above and/or below the bone. Typically, these are considered nails/screws/blades where insertion requires pushing back on the interbody device to include hammering/screwing/drilling toward the patient's spinal cord. Most often these devices catch/pass through the front of the interbody device although some are spikes that are further back. The risk of dislodging the interbody device with this technique may lead to damage to the spinal cord that sits in-line with the force being applied to the graft or spacer. Another difficulty with this traditional stand-alone device can be the inability of “recessing” the front portion of this device as the “locking” portals are blocked. Specifically, fasteners that are inserted into these devices must be introduced at an angle to a horizontal plane and recessing the implant beyond an anterior surface of the vertebral bodies blocks both the insertion and removal of these angled fasteners into the anterior surface of these implants and upwardly or downwardly into the vertebral bodies. In addition, the anterior placement of many of these “stand alone” devices can result in patient complications related to fracturing or shearing off front or anterior portions of the vertebral bodies from the spikes/blades/screws.

Furthermore, these multiple solid locking mechanisms do not promote boney growth into the locking mechanism or the interbody device.

In addition, these multiple solid locking mechanisms do not provide “counter” control while being engaged to prevent movement of the interbody device. These current locking mechanisms also do not allow for boney growth through the mechanism and the industry is generally lacking stacked mechanisms. The prior art further does not facilitate mounting of the fastening mechanism to the spacer after the spacer is implanted between the two bones.

It would be desirable to design, develop, construct and deploy and interbody spacer that addresses the deficiencies of the known interbody implants.

### BRIEF SUMMARY OF THE INVENTION

Briefly stated, the preferred invention is directed to a locking mechanism for joining bones or bone fragments that may be implemented with an interbody spacer for promoting fusion between first and second vertebral bodies. The implant or interbody spacer includes a peripheral wall defining an internal space and a rotatable fastener connected to the peripheral wall. A lattice or web is positioned within the peripheral wall that includes a relief space to facilitate the rotatable fastener. The rotatable fastener includes a shaft, a first blade and a second blade. The rotatable fastener is movable from an insertion configuration to an expanded configuration. The first and second blades are positioned between upper and lower planes defined by the peripheral wall in the insertion configuration and extend beyond the upper and lower planes in the expanded configuration.

Depending which embodiment is looked at, the preferred ax fastener or locking mechanism connects the two adjacent bones, preferably endplates of vertebrae, above and below as a functioning device that functions nearly independent of the implant or interbody device and promotes fusion between the bones. Other stand-alone technology fixation devices fix the endplates to the interbody device. The prior art devices screw through the interbody and then into the endplate or adjacent bone. Embodiments of the preferred ax fastener or locking mechanism extends between the two adjacent endplates or bone portions and through the implant or interbody spacer. The preferred implant with the open architecture, web or lattice promotes a second level of fusion across the fusion joint or disc space because the implant captures bone not just between the two endplates but by pulling or “sewing” the bone/interbody space together. These double fusion opportunities are not facilitated by prior art devices.

### BRIEF DESCRIPTION OF THE SEVERAL VIEWS OF THE DRAWINGS

The foregoing summary, as well as the following detailed description of preferred embodiments of the instrument, implant and method of the present application, will be better understood when read in conjunction with the appended drawings. For the purposes of illustrating the locking mechanism for the orthopedic implant or interbody spacer, there are shown in the drawings preferred embodiments. It should be understood, however, that the application is not limited to the precise arrangements and instrumentalities shown. In the drawings:

FIG. 1A illustrates a front elevational view of an implant or interbody spacer with a fastener or locking mechanism in accordance with a first preferred embodiment of the present invention in a mounted and expanded configuration;

FIG. 1B illustrates a sagittal cross-sectional view of the implant or interbody spacer with the fastener of FIG. 1A, taken along line 1B-1B of FIG. 1A;

FIG. 2A illustrates a top perspective view of the implant or interbody spacer with the fastener of FIG. 1A in an insertion configuration;

FIG. 2B illustrates a top plan view of the implant or interbody spacer with the fastener of FIG. 1A in the insertion configuration;

FIG. 2C illustrates a front elevational view of the implant or interbody spacer with the fastener of FIG. 1A in the insertion configuration;

FIG. 3A illustrates an alternative top perspective view of the implant or interbody spacer with the fastener of FIG. 1A in the expanded configuration;

FIG. 3B illustrates an alternative top plan view of the implant or interbody spacer with the fastener of FIG. 1A in the expanded configuration;

FIG. 3C illustrates an alternative front elevational view of the implant or interbody spacer with the fastener of FIG. 1A, in the expanded configuration;

FIG. 4A illustrates another alternative top plan view of the implant or interbody spacer with the fastener of FIG. 1A, wherein a length of the blades may be expanded to facilitate additional purchase into the bones;

FIG. 4B illustrates another front elevational view of the implant or interbody spacer with the fastener of FIG. 1A;

FIG. 5A illustrates an additional alternative top perspective view of the implant or interbody spacer with the fastener of FIG. 1A, wherein the shaft and blades are mobile upon manufacture and insertion to into the slot but fixed upon rotation and engagement with the adjacent bones;

FIG. 5B illustrates an additional alternative top plan view of the implant or interbody spacer with the fastener of FIG. 1A;

FIG. 6A illustrates a top perspective view of an implant or interbody spacer with the fastener in accordance with a second preferred embodiment of the present invention;

FIG. 6B illustrates a front elevational view of the implant or interbody spacer with the fastener of FIG. 6A;

FIG. 7A illustrates a top perspective view of an implant or interbody spacer with the fastener in accordance with a third preferred embodiment of the present invention;

FIG. 7B illustrates a top plan view of the implant or interbody spacer with the fastener of FIG. 7A;

FIG. 8A illustrates a top perspective view of an implant or interbody spacer with the fastener in accordance with a fourth preferred embodiment of the present invention;

FIG. 8B illustrates a top plan view of the implant or interbody spacer with the fastener of FIG. 8A;

FIG. 9A illustrates top perspective view of an implant or interbody spacer with the fastener in accordance with a fifth preferred embodiment of the present invention;

FIG. 9B illustrates a top plan view of the implant or interbody spacer with the fastener of FIG. 9A;

FIG. 10A illustrates a top perspective view of an implant or interbody spacer with the fastener in accordance with a sixth preferred embodiment of the present invention, wherein the blade may be pre-assembled on the peripheral wall or may be assembled to the peripheral wall after the peripheral wall is implanted between the bones;

FIG. 10B illustrates a top plan view of the implant or interbody spacer with the fastener of FIG. 10A;

FIG. 11A illustrates a top perspective view of an implant or interbody spacer with the fastener in accordance with a seventh preferred embodiment of the present invention;

FIG. 11B illustrates a top plan view of the implant or interbody spacer with the fastener of FIG. 11A;

FIG. 12A illustrates a top perspective view of an implant or interbody spacer with the fastener in accordance with an eighth preferred embodiment of the present invention;

FIG. 12B illustrates a top plan view of the implant or interbody spacer with the fastener of FIG. 12A;

FIG. 13A illustrates a top plan view of a fastening blade in accordance with a preferred embodiment of the present invention, wherein the blade has a lattice or web configuration that facilitates bone growth into and through the blade in an expanded configuration;

FIG. 13B illustrates a top plan view of a fastening blade in accordance with another preferred embodiment of the present invention;

FIG. 14A illustrates a top perspective view of an implant or interbody spacer with the fastener in accordance with a ninth preferred embodiment of the present invention, wherein the implant or interbody spacer with the fastener is in an insertion configuration;

FIG. 14B illustrates a front elevational view of the implant or interbody spacer with the fastener of FIG. 14A;

FIG. 14C illustrates a top perspective view of the implant or interbody spacer with the fastener of FIG. 14A in an expanded configuration, wherein the implant or interbody spacer with the fastener is in an elevated configuration;

FIG. 14D illustrates a front elevational view of the implant or interbody spacer with the fastener of FIG. 14A in the expanded configuration;

FIG. 15A illustrates a top perspective view of an implant or interbody spacer with the fastener in accordance with a tenth preferred embodiment of the present invention;

FIG. 15B illustrates a front elevational view of the implant or interbody spacer with the fastener of FIG. 15A;

FIG. 15C illustrates a top perspective view of the implant or interbody spacer with the fastener of FIG. 15A in an expanded configuration;

FIG. 15D illustrates a front elevational view of the implant or interbody spacer with the fastener of FIG. 15A in the expanded configuration;

FIG. 16A illustrates a top perspective view of an implant or interbody spacer with the fastener in accordance with an eleventh preferred embodiment of the present invention;

FIG. 16B illustrates a top plan view of the implant or interbody spacer with the fastener of FIG. 16A;

FIG. 16C illustrates a side elevational view of the implant or interbody spacer with the fastener of FIG. 16A;

FIG. 17A illustrates a side elevational view of the implant or interbody spacer with the fastener of FIG. 1A and an insertion tool;

FIG. 17B illustrates a side elevational view of the implant or interbody spacer with the fastener of FIG. 1A, the insertion tool of FIG. 17A and an intermediate fastening tool;

FIG. 18 illustrates a top plan view of the implant or interbody spacer with the fastener and insertion tool of FIG. 17A;

FIG. 19A illustrates a front elevation view of another embodiment with one blade parallel to the front of the implant;

FIG. 19B illustrates a top elevation view of FIG. 19A;

FIG. 19C illustrates a top elevation perspective view of FIG. 19A;

FIG. 19D illustrates a side elevation view of FIG. 19A;

FIG. 19E illustrates a bottom elevation view of FIG. 19A;

FIG. 19F illustrates a bottom elevation view of FIG. 19A with the blade shown in fantom lines;

FIG. 19G illustrates an end view of FIG. 19A with the blade shown in fantom lines;

FIG. 19H illustrates a side view of FIG. 19A with the blade shown in fantom lines;

FIG. 19I illustrates a perspective view of FIG. 19A with the blade shown in fantom lines;

FIG. 19J illustrates a perspective end view of FIG. 19A with the blade shown in fantom lines;

FIG. 20A illustrates a front elevation view of another embodiment with one deployed blade perpendicular to the front of the implant;

FIG. 20B illustrates an end view of FIG. 20A with a deployed blade;

FIG. 20C illustrates a side elevation view of FIG. 20A with a deployed blade;

FIG. 20D illustrates a perspective elevation view of

FIG. 20A with a deployed blade;

FIG. 20E illustrates a bottom view of FIG. 20A with the blade in fantom lines;

FIG. 20F illustrates a side elevation view of FIG. 20A with the blade retracted in fantom lines;

FIG. 20G illustrates a side view of FIG. 20A;

FIG. 20H illustrates a perspective view of FIG. 20A with the blade shown in fantom lines;

FIG. 21A illustrates a front elevation view of another embodiment with two blades parallel to the front of the implant;

FIG. 21A illustrates a front elevation view of another embodiment with one deployed blade perpendicular to the front of the implant;

FIG. 21B illustrates a side view of FIG. 21A with two deployed blades;

FIG. 21C illustrates a perspective view of FIG. 21A with deployed blades;

FIG. 21D illustrates a front end perspective elevation view of FIG. 21A with deployed blades;

FIG. 21E illustrates a back end perspective elevation view of FIG. 21A with deployed blades;

FIG. 21F illustrates a back end view of FIG. 21A with the blades retracted in fantom lines;

FIG. 21G illustrates a side view of FIG. 21F;

FIG. 21H illustrates a perspective view of FIG. 21F with the blades shown in fantom lines;

FIG. 21I illustrates a perspective view of FIG. 21F with the blades shown in fantom lines;

FIG. 22A illustrates a front elevation view of another embodiment with an offset (off center) blade parallel to the front of the implant;

FIG. 22B illustrates a side view of FIG. 22A with a deployed blade;

FIG. 22C illustrates a perspective view of FIG. 22A with a deployed blade;

FIG. 22D illustrates a front end perspective elevation view of FIG. 22A with a deployed blade;

FIG. 22E illustrates a back end perspective elevation view of FIG. 22A with a deployed blade;

FIG. 22F illustrates a back end view of FIG. 22A with the blade retracted in fantom lines;

FIG. 22G illustrates a side view of FIG. 22F;

FIG. 22H illustrates a perspective view of FIG. 22F with the blade shown in fantom lines;

FIG. 22I illustrates a perspective view of FIG. 22F with the blade shown in fantom lines; and

FIG. 22J illustrates a perspective view of FIG. 22F with the blade shown in phantom lines.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred implant or interbody spacer with the fastener and related parts thereof. The words, “anterior”, “posterior”, “superior,” “inferior”, “lateral” and related words and/or phrases designate preferred positions, directions and/or orientations in the human body to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

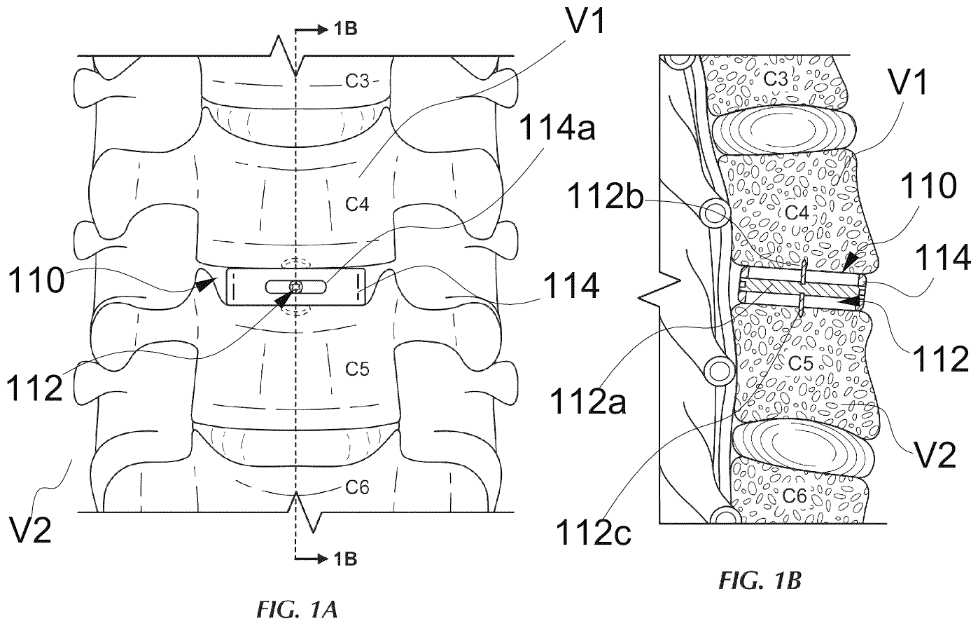

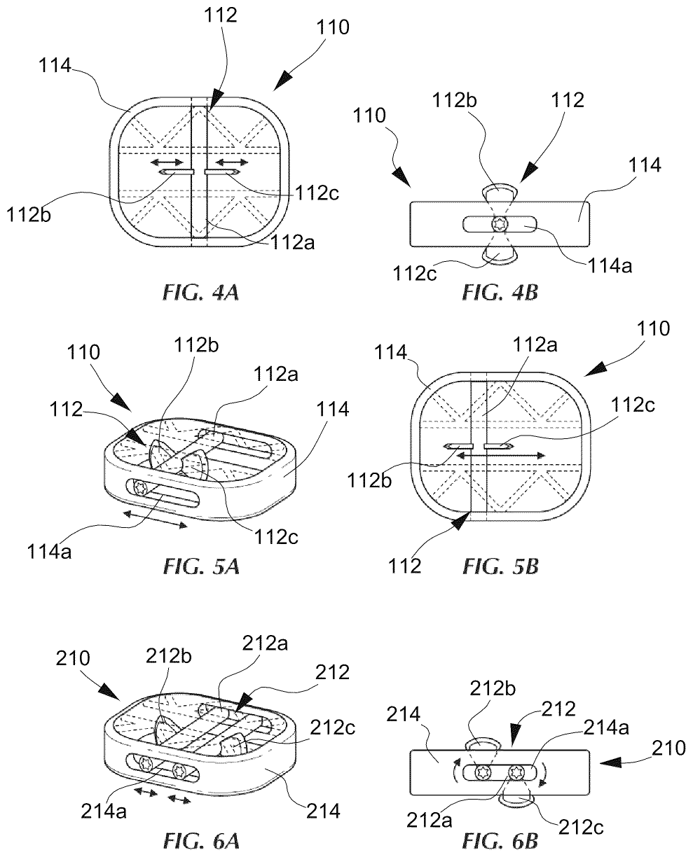

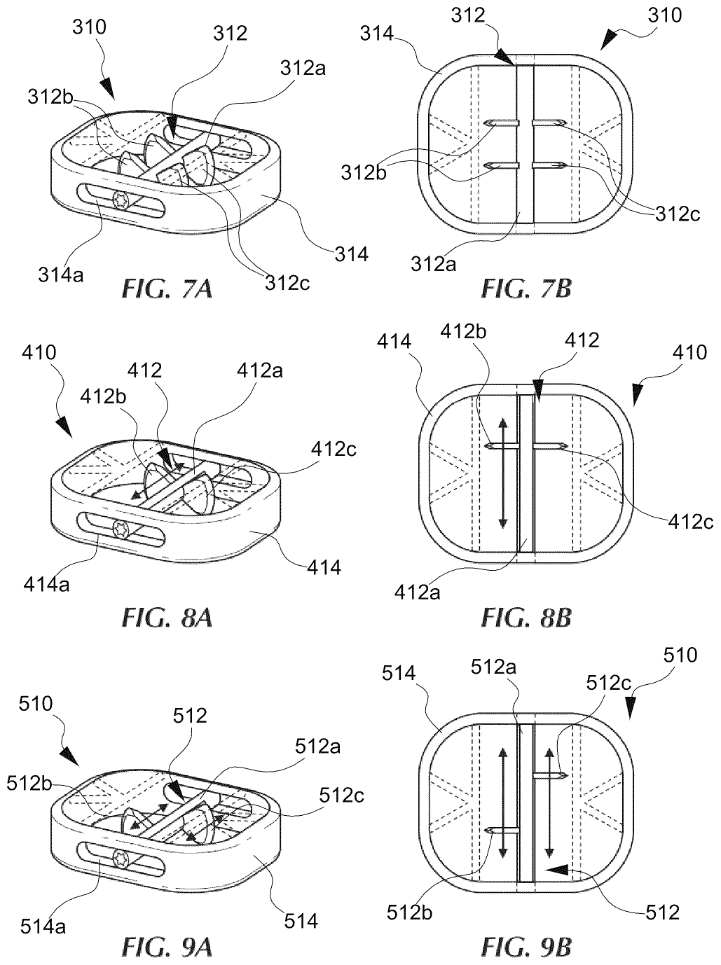

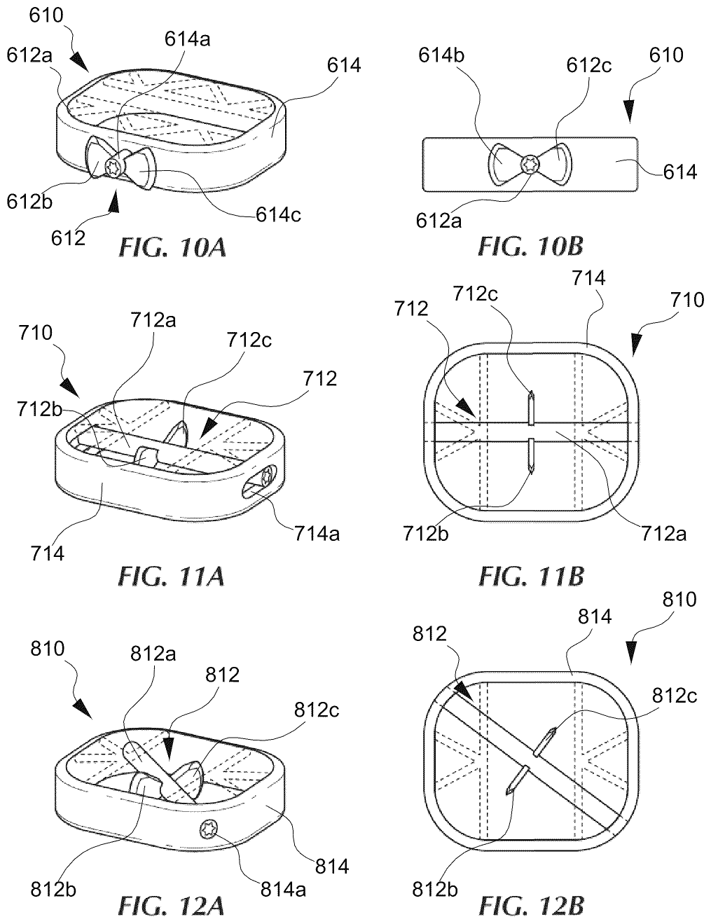

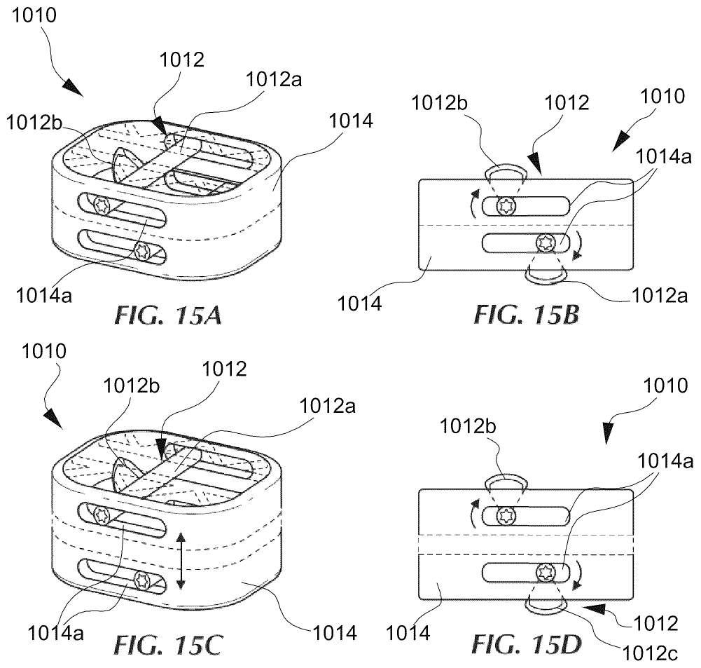

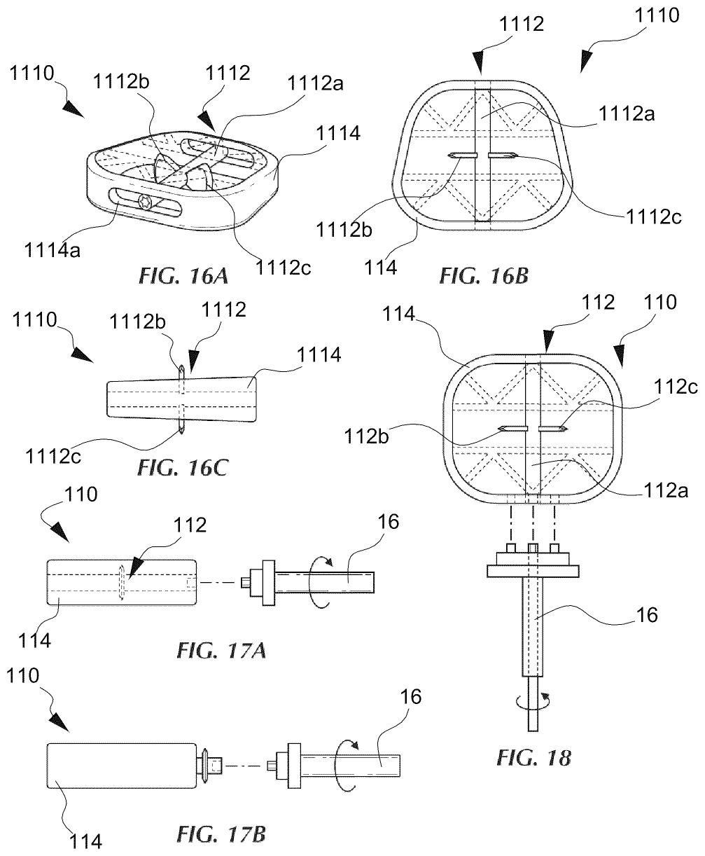

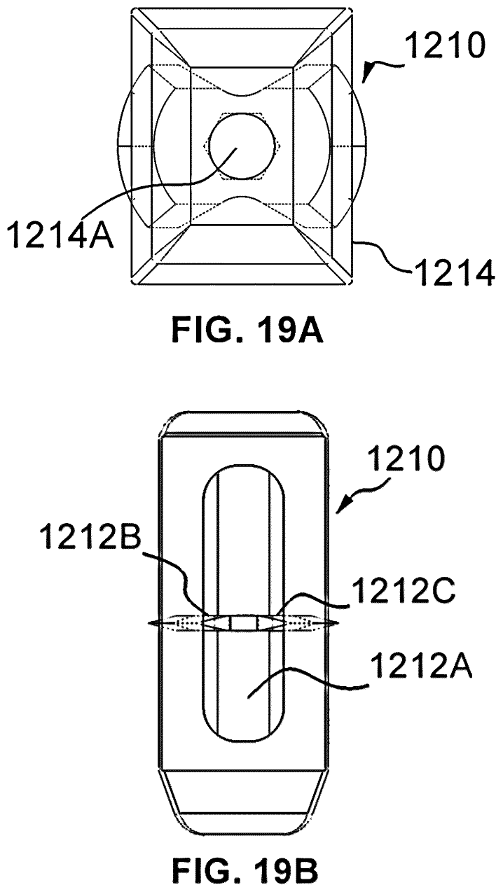

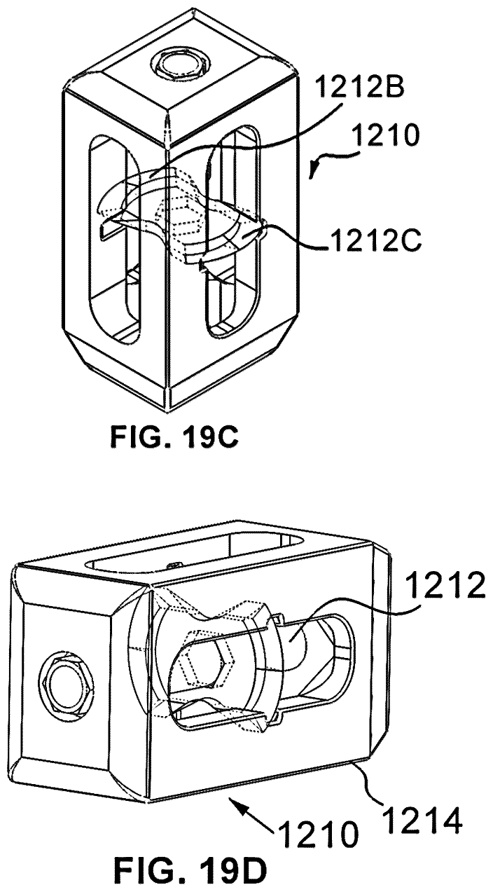

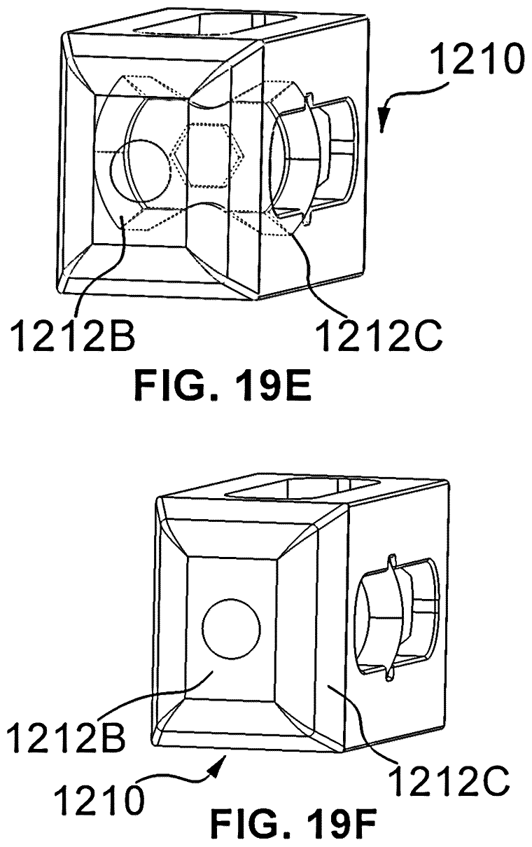

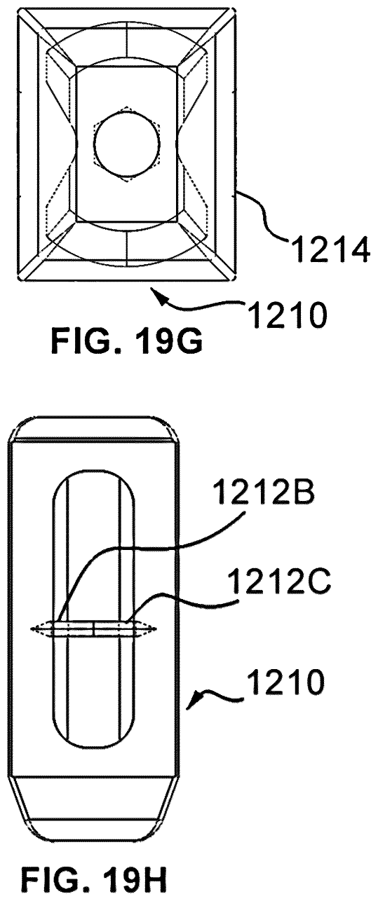

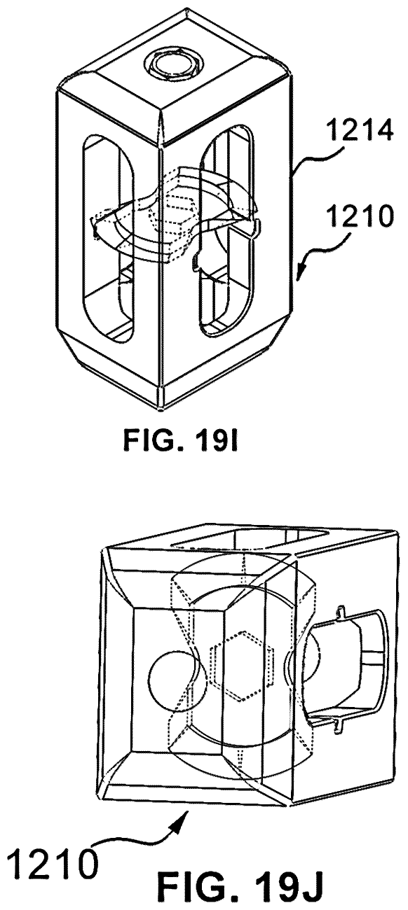

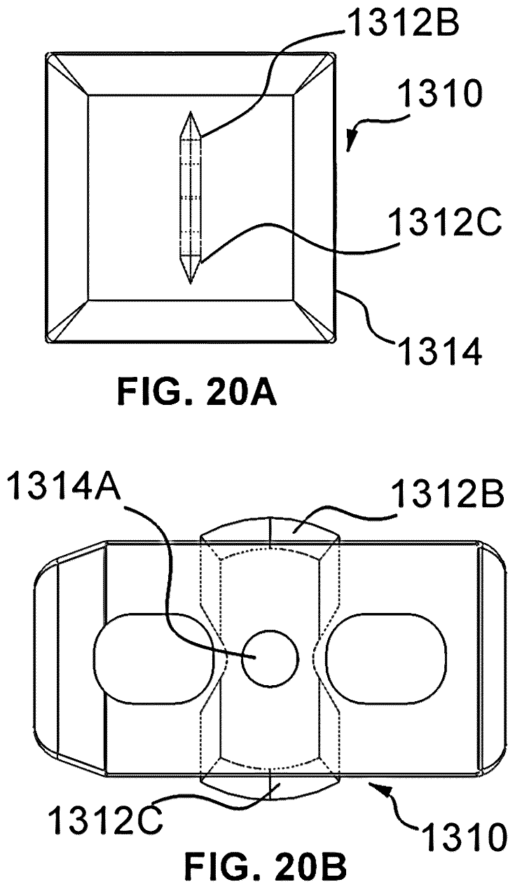

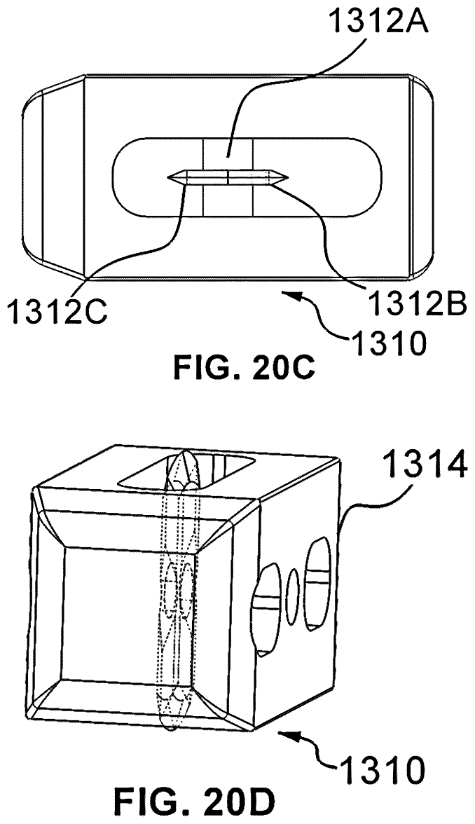

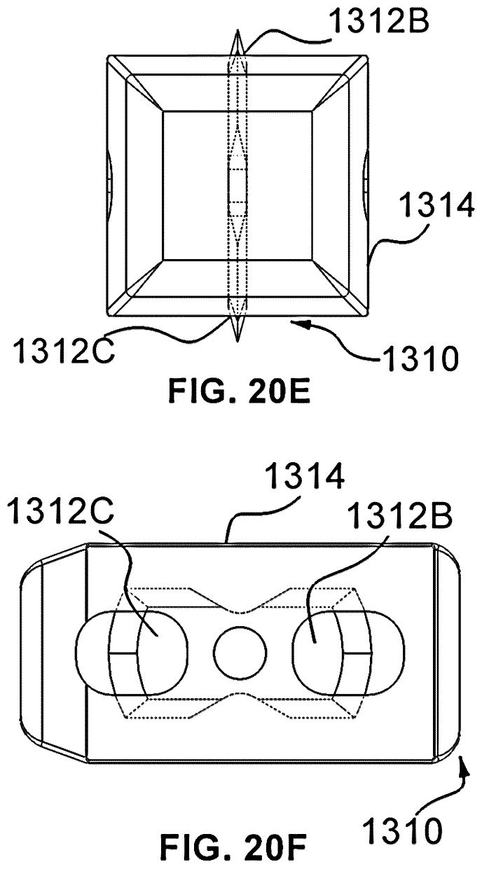

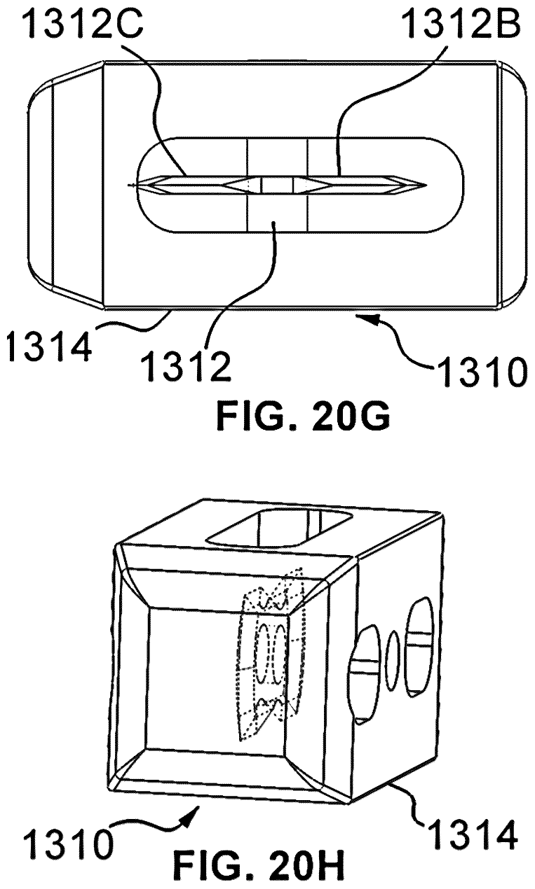

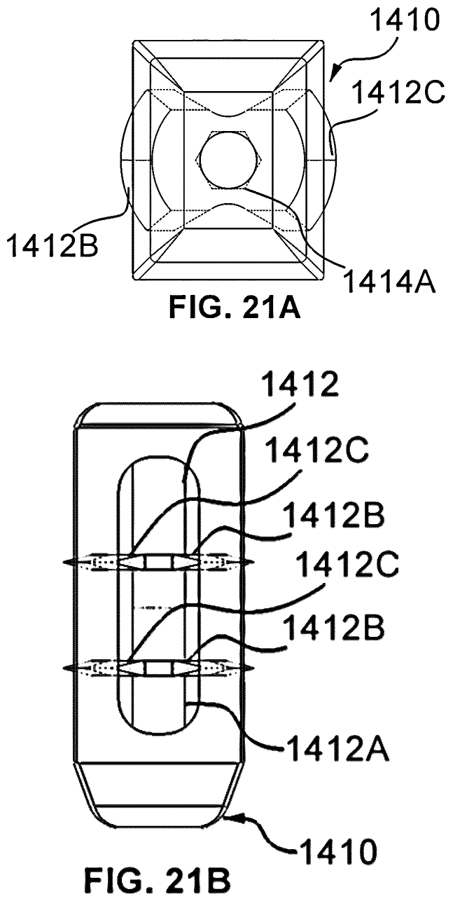

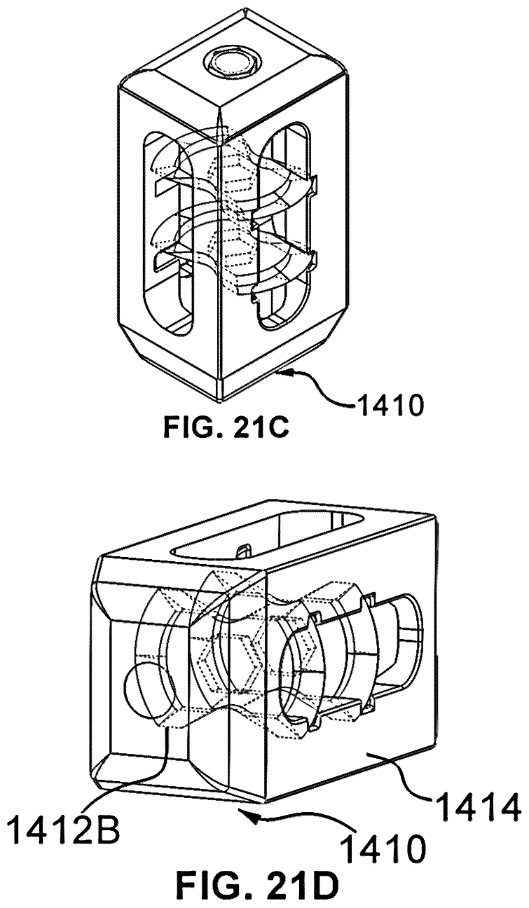

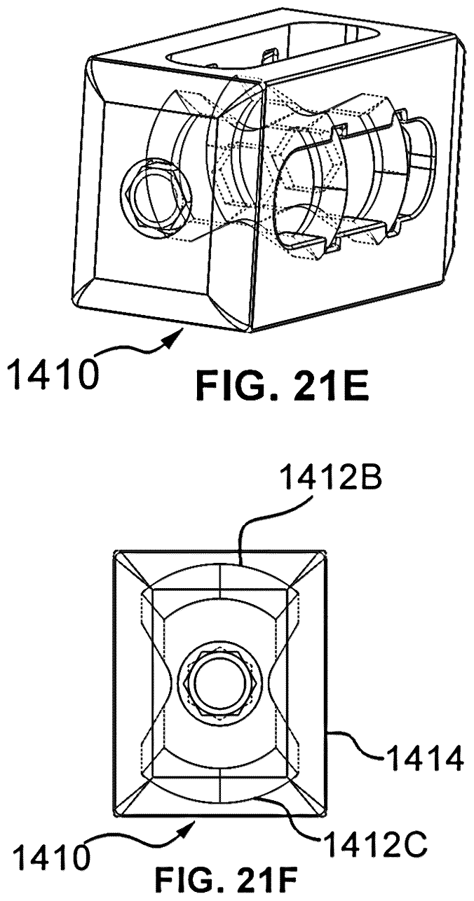

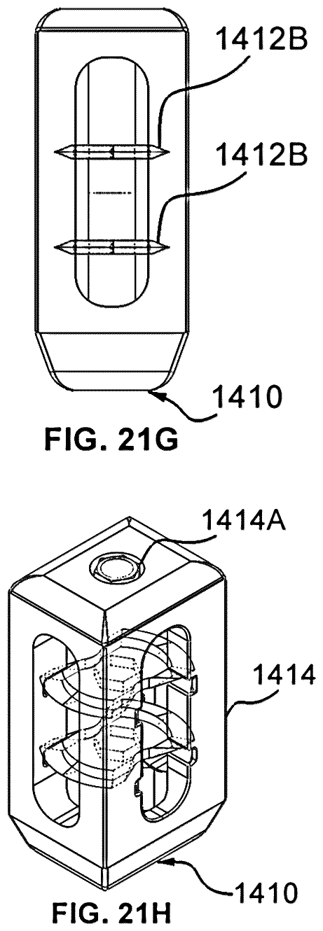

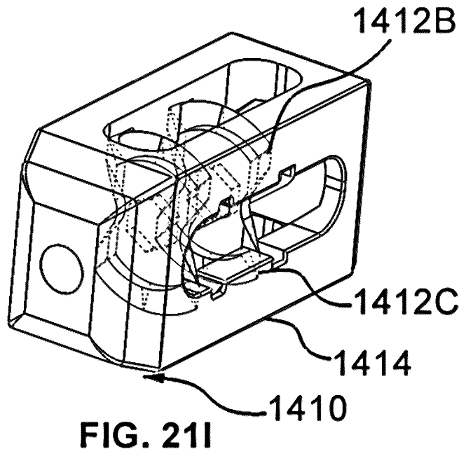

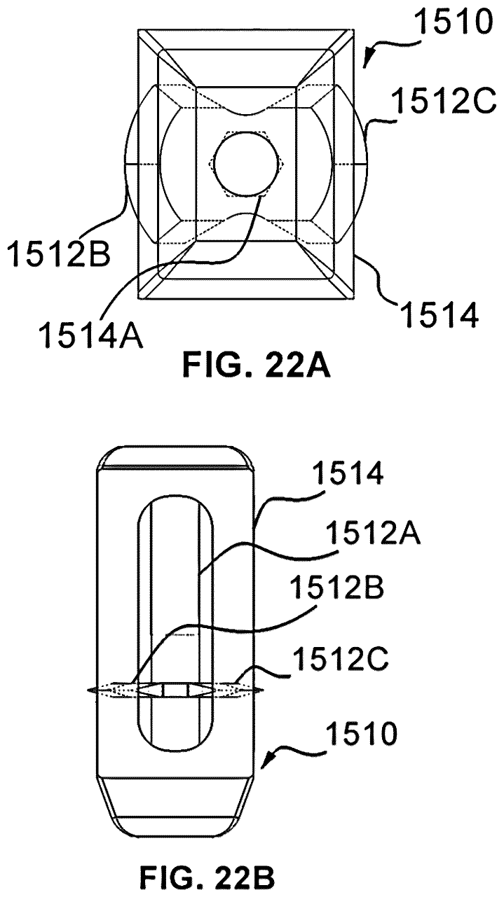

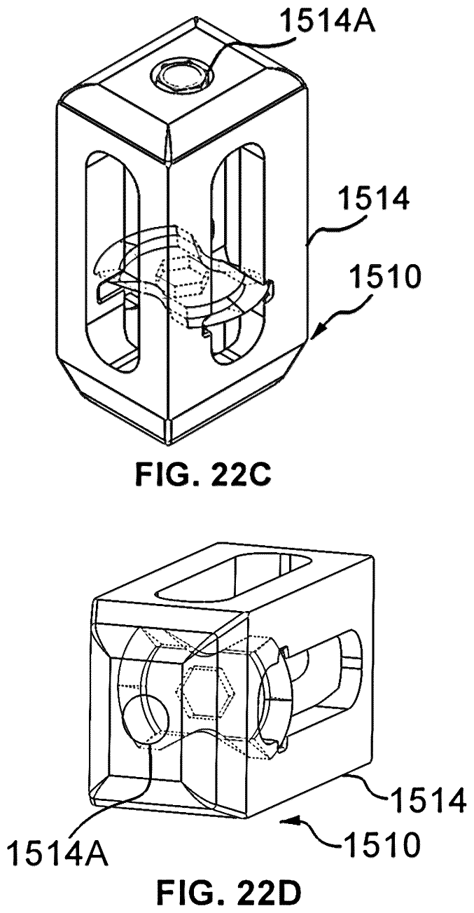

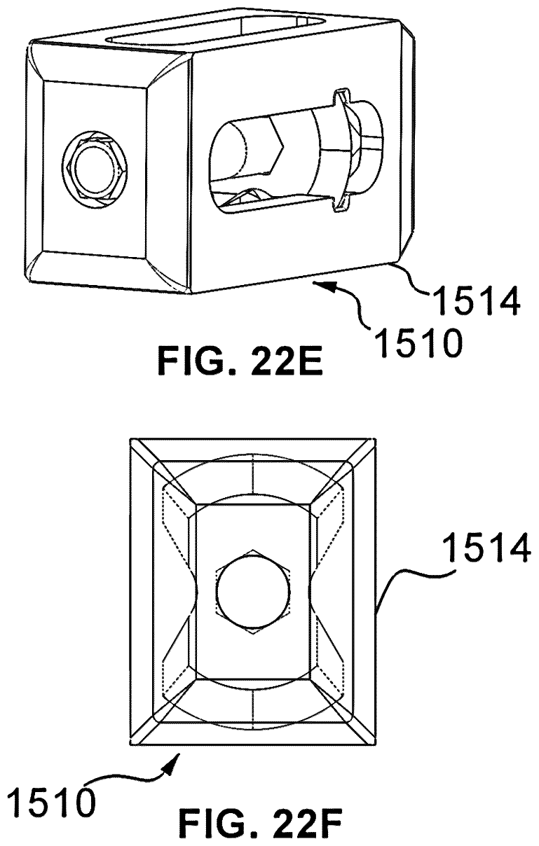

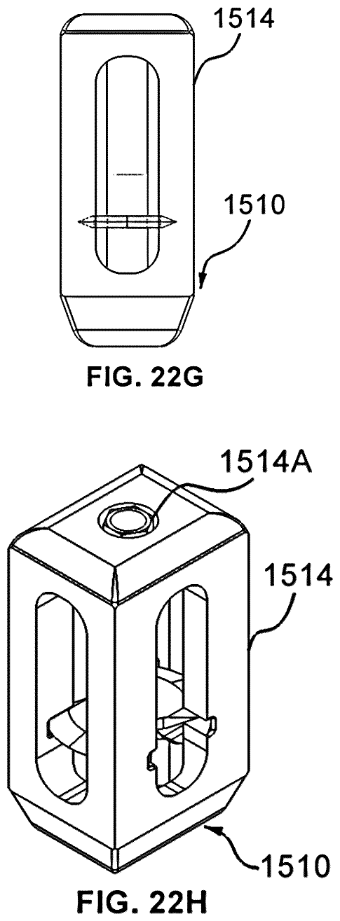

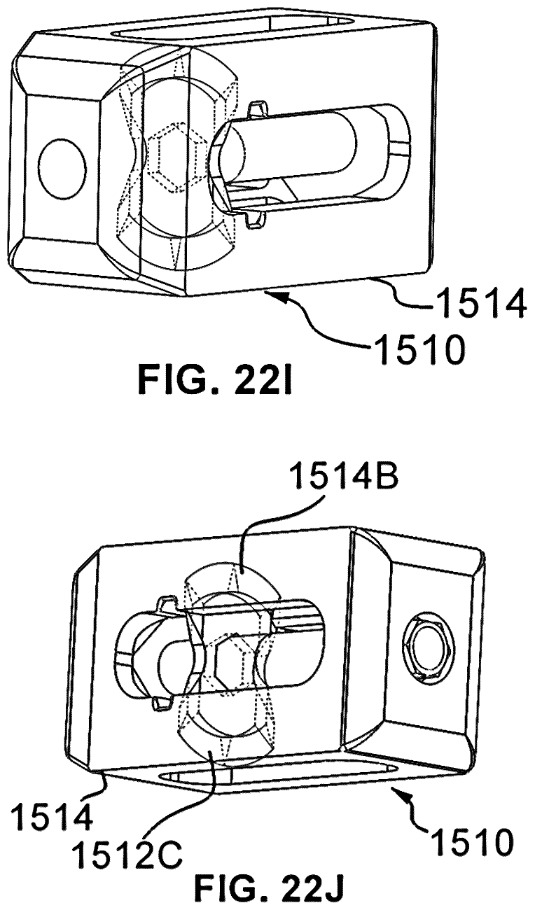

Referring to FIGS. 1-18, first, second, third, fourth, fifth, sixth, seventh, eighth, ninth, tenth and eleventh preferred embodiments of an spacer, implant or interbody generally designated 110, 210, 310, 410, 510, 610, 710, 810, 910, 1010, 1110, 1210, 1310, 1410, 1510, are generally used for promoting fusion between superior and inferior vertebral bodies V1, V2. The preferred interbody spacers 110, 210, 310, 410, 510, 610, 710, 810, 910, 1010, 1110, 1210, 1310, 1410, 1510 are typically positioned between the vertebral bodies V1, V2 to facilitate fusion of the two vertebral bodies V1, V2 and are secured to the vertebral bodies V1, V2 by an ax fastener or locking mechanism 112, 212, 312, 412, 512, 612, 712, 812, 912, 1012, 1112, 1212, 1312, 1412, 1512. The preferred implant or interbody spacers 110, 210, 310, 410, 510, 610, 710, 810, 910, 1010, 1110, 1210, 1310, 1410, 1510 include similar features and constructions when compared to each other. The same reference numerals are utilized to identify similar features of the various preferred embodiments when compared to each of the preferred interbody spacers 110, 210, 310, 410, 510, 610, 710, 810, 910, 1010, 1110, 1210, 1310, 1410, 1510 with a “1” prefix to distinguish the first preferred embodiment of the interbody spacer 110, a “2” prefix to distinguish the second preferred embodiment of the interbody spacer 210, a “3” prefix to distinguish the third preferred embodiment of the interbody spacer 310, a “4” prefix to distinguish the fourth preferred embodiment of the interbody spacer 410, a “5” prefix to distinguish the fifth preferred embodiment of the interbody spacer 510, a “6” prefix to distinguish the sixth preferred embodiment of the interbody spacer 610, a “7” prefix to distinguish the seventh preferred embodiment of the interbody spacer 710, an “8” prefix to distinguish the eighth preferred embodiment of the interbody spacer 810, a “9” prefix to distinguish the ninth preferred embodiment of the interbody spacer 910, a “10” prefix to distinguish the tenth preferred embodiment of the interbody spacer 1010; an “11” prefix to distinguish the eleventh preferred embodiment of the interbody spacer 1110; a “12” prefix to distinguish the twelfth preferred embodiment of the interbody spacer 1210; a “13” prefix to distinguish the thirteenth preferred embodiment of the interbody spacer 1310; a “14” prefix to distinguish the fourteenth preferred embodiment of the interbody spacer 1410; and a “15” prefix to distinguish the fifteenth preferred embodiment of the interbody spacer 1510.

The preferred implants or interbody spacers 110, 210, 310, 410, 510, 610, 710, 810, 910, 1010, 1110, 1210, 1310, 1410, 1510 include a peripheral wall 114, 214, 314, 414, 514, 614, 714, 814, 914, 1014, 1114, 1214, 1314, 1414, 1514 and a pivotable locking mechanism or ax fastener 112, 212, 312, 412, 512, 612, 712, 812, 912, 1012, 1112, 1212, 1312, 1412, 1512 that is pivotably mounted to the peripheral wall 114, 214, 314, 414, 514, 614, 714, 814, 914, 1014, 1114, 1214, 1314, 1414, 1514. The ax fastener 112, 212, 312, 412, 512, 612, 712, 812, 912, 1012, 1112, 1212, 1312, 1412, 1512 includes a shaft 112a, 212a, 312a, 412a, 512a, 612a, 712a, 812a, 912a, 1012a, 1112a, 1212a, 1312a, 1412a, 1512a and first and second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that are secured to the shaft 112a, 212a, 312a, 412a, 512a, 612a, 712a, 812a, 912a, 1012a, 1112a, 1212a, 1312a, 1412a, 1512a. The shaft 112a, 212a, 312a, 412a, 512a, 712a, 912a, 1012a, 1112a, 1212a, 1312a, 1412a, 1512a is preferably mounted in a slot 114a, 214a, 314a, 414a, 514a, 714a, 914a, 1014a, 1114a, 1214a, 1314a, 1414a, 1514a in the peripheral wall 114, 214, 314, 414, 514, 614, 714, 814, 914, 1014, 1114, 1214, 1314, 1414, 1514 that permits lateral movement of the shaft 112a, 212a, 312a, 412a, 512a, 712a, 912a, 1012a, 1112a, 1212a, 1312a, 1412a, 1512a relative to the peripheral wall 114, 214, 314, 414, 514, 614, 714, 814, 914, 1014, 1114, 1214, 1314, 1414, 1514, but is not so limited and, for example, may be mounted in holes 614a, 814a in the peripheral wall 614, 814 of the sixth and eighth preferred embodiments that generally fix the position of the shaft 612, 812 relative to the peripheral wall 614, 814 but permit pivoting or rotation of the shaft 612, 812 relative to the peripheral wall 614, 814. The slots 114a, 214a, 314a, 414a, 514a, 714a, 914a, 1014a, 1114a, 1214a, 1314a, 1414a, 1514a in the peripheral wall 114, 214, 314, 414, 514, 614, 714, 814, 914, 1014, 1114, 1214, 1314, 1414, 1514, as well as a channel that extends into and through the lattice within the peripheral wall 114, 214, 314, 414, 514, 614, 714, 814, 914, 1014, 1114, 1214, 1314, 1414, 1514 facilitate mounting of the shaft 112a, 212a, 312a, 412a, 512a, 712a, 912a, 1012a, 1112a, 1212a, 1312a, 1412a, 1512a and the first and second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c to the peripheral wall 114, 214, 314, 414, 514, 614, 714, 814, 914, 1014, 1114, 1214, 1314, 1414, 1514 after the peripheral wall 114, 214, 314, 414, 514, 614, 714, 814, 914, 1114, 1214, 1314, 1414, 1514 is implanted between the vertebral bodies V1, V2. The channel and the slots 114a, 214a, 314a, 414a, 514a, 714a, 914a, 1014a, 1114a, 1214a, 1314a, 1414a, 1514a are preferably designed to permit sliding or movement of the shaft 112a, 212a, 312a, 412a, 512a, 712a, 912a, 1012a, 1112a, 1212a, 1312a, 1412a, 1512a and the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c into position relative to the peripheral wall 114, 214, 314, 414, 514, 614, 714, 814, 914, 1014, 1114, 1214, 1314, 1414, 1514 after the peripheral wall 114, 214, 314, 414, 514, 614, 714, 814, 914, 1014, 1114, 1214, 1314, 1414, 1514 and lattice are implanted between the vertebral bodies V1, V2, as well as rotation of the shaft 112a, 212a, 312a, 412a, 512a, 712a, 912a, 1012a, 1112a, 1212a, 1312a, 1412a, 1512a that moves the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c from the insertion to the expanded configuration.

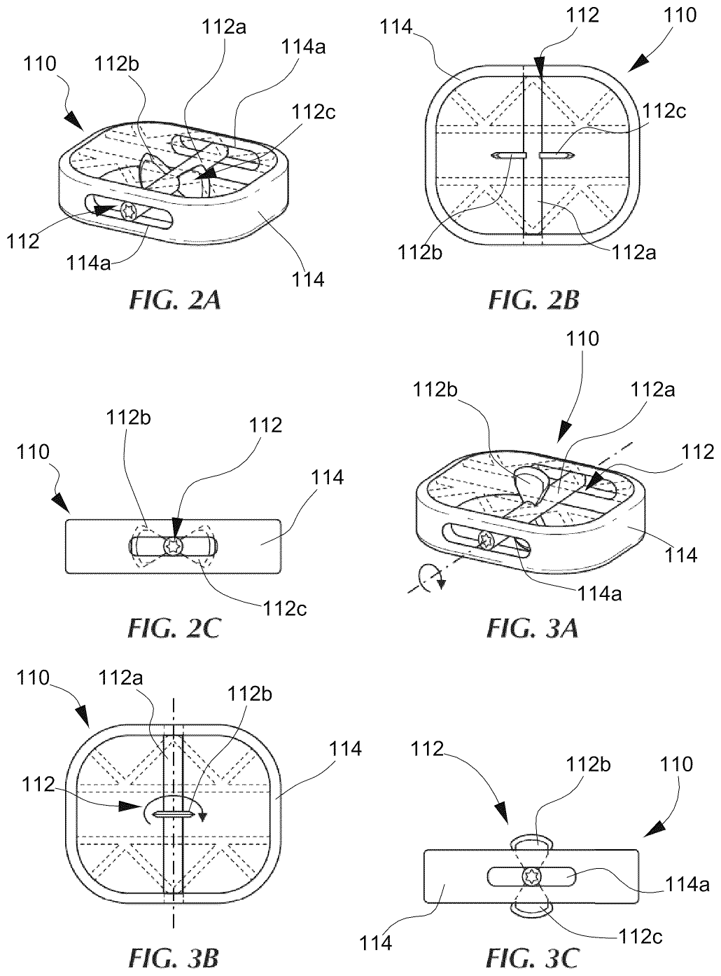

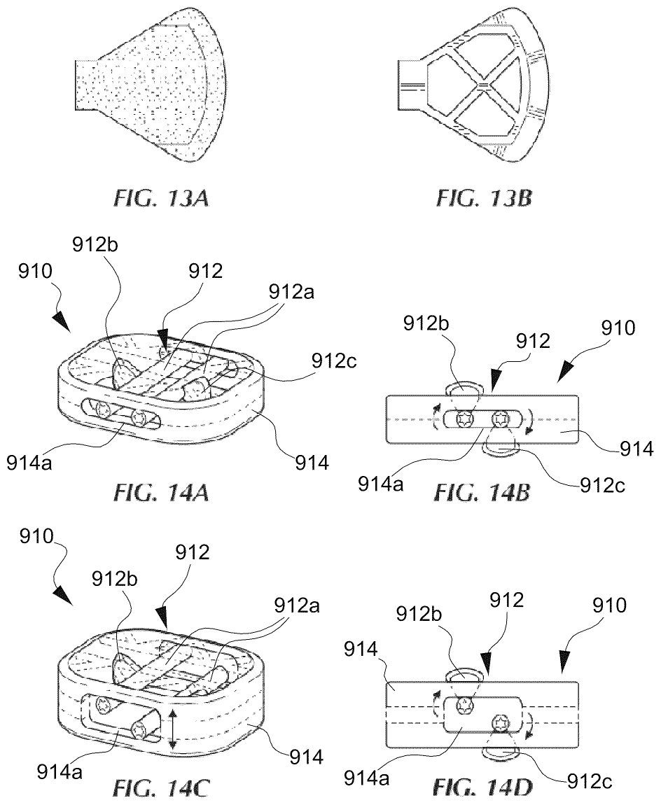

The first and second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be positioned at nearly any location on the shaft 112a, 212a, 312a, 412a, 512a, 612a, 712a, 812a, 912a, 1012a, 1112a, 1212a, 1312a, 1412a, 1512a and/or on any location on the peripheral wall 114, 214, 314, 414, 514, 614, 714, 814, 914, 1014, 1114, 1214, 1314, 1414, 1514 or on other locations on the interbody spacer 110, 210, 310, 410, 510, 610, 710, 810, 910, 1010, 1110, 1210, 1310, 1410, 1510. The first or second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be anywhere on the perimeter or interior of the interbody spacer 110, 210, 310, 410, 510, 610, 710, 810, 910, 1010, 1110, 1210, 1310, 1410, 1510 based on designer and/or surgeon preferences. A single blade (FIGS. 13A and 13B) may be placed anywhere on the rotatable shaft 112a, 212a, 312a, 412a, 512a, 612a, 712a, 812a, 912a, 1012a, 1112a, 1212a, 1312a, 1412a, 1512a or may be configured as a double blade, as is shown in FIGS. 2A-12B and 14A-18. When the single blade is utilized, the single blade preferably engages one endplate of the vertebral bodies V1, V2 in the mounted configuration. Single blades may be staggered along the rotatable shaft 112a, 212a, 312a, 412a, 512a, 612a, 712a, 812a, 912a, 1012a, 1112a, 1212a, 1312a, 1412a, 1512a (FIGS. 9A and 9B). The single blade configuration may allow stabilization of the interbody spacer 110, 210, 310, 410, 510, 610, 710, 810, 910, 1010, 1110, 1210, 1310, 1410, 1510 to both adjacent vertebral bodies V1, V2 or just one of the vertebral bodies V1, V2. In the “double blade” configuration, the preferred interbody spacer 110, 210, 310, 410, 510, 610, 710, 810, 910, 1010, 1110, 1210, 1310, 1410, 1510 spans the motion segment providing stabilization by both connecting to the interbody spacer 110, 210, 310, 410, 510, 610, 710, 810, 910, 1010, 1110, 1210, 1310, 1410, 1510, as well as by uniquely connecting the adjacent vertebral bodies V1, V2.

The Viking locking blade, ax mechanism or pivotable ax fastener 112, 212, 312, 412, 512, 612, 712, 812, 912, 1012, 1112, 1212, 1312, 1412, 1512 may be designed and configured for positioning anywhere in the interbody spacer 110, 210, 310, 410, 510, 610, 710, 810, 910, 1010, 1110, 1210, 1310, 1410, 1510 and, particularly, relative to the peripheral wall 114, 214, 314, 414, 514, 614, 714, 814, 914, 1014, 1114, 1214, 1314, 1414, 1514 from front to back as well as side to side. The pivotable ax fastener 112, 212, 312, 412, 512, 612, 712, 812, 912, 1012, 1112, 1212, 1312, 1412, 1512 may be designed and configured to be positioned on any rotational axis relative to the peripheral wall 114, 214, 314, 414, 514, 614, 714, 814, 914, 1014, 1114, 1214, 1314, 1414, 1514 before deployed such that the first and second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1211c, 1311c, 1411, and 1511c extend above and below upper and lower surfaces or planes of the peripheral wall 114, 214, 314, 414, 514, 614, 714, 814, 914, 1014, 1114, 1214, 1314, 1414, 1514 and into the vertebral bodies V1, V2 in the mounted configuration.

The ax fastener 112, 212, 312, 412, 512, 612, 712, 812, 912, 1012, 1112, 1212, 1312, 1412, 1512 may be comprised of a double (2 sided) solid blade, including the first and second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c, with a single shaft 112a, 212a, 312a, 412a, 512a, 612a, 712a, 812a, 912a, 1012a, 1112a, 1212a, 1312a, 1412a and 1512a for rotation and extension of portions of the first and second blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c above and below the interbody device or the upper and lower planes of the peripheral wall 114, 214, 314, 414, 514, 614, 714, 814, 914, 1014, 1114, 1214, 1314, 1414, 1514 to engage the vertebral bodies V1, V2. The ax fastener 112, 212, 312, 412, 512, 612, 712, 812, 912, 1012, 1112, 1212, 1312, 1412, 1512 may also be constructed with one or multiple individual single blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that are independent or coordinated with screw holes to rotate the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c above/below and outside the interbody or index implant or interbody spacer 110, 210, 310, 410, 510, 610, 710, 810, 910, 1010, 1110, 1210, 1310, 1410, 1510.

The blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may start positioned within the interior of the interbody spacer 110, 210, 310, 410, 510, 610, 710, 810, 910, 1010, 1110, 1210, 1310, 1410, 1510 in an insertion configuration (FIGS. 2A-3C, 4A, 5A, 6A, 7A-12B, 14A within the peripheral walls 114, 214, 314, 414, 514, 614, 714, 814, 914, 1014, 1114, 1214, 1314, 1414, 1514 of the index implant or the interbody spacer 110, 210, 310, 410, 510, 610, 710, 810, 910, 1010, 1110, 1210, 1310, 1410, 1510 and may be actuated to an expanded configuration such that portion of the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c extend outside the perimeter of the peripheral walls 114, 214, 314, 414, 514, 614, 714, 814, 914, 1014, 1114, 1214, 1314, 1414, 1514 of the index implant or the interbody spacer 110, 210, 310, 410, 510, 610, 710, 810, 910, 1010, 1110, 1210, 1310, 1410, 1510.

The blades 112b, 212b, 312b, 412b, 512b, 612b, 712b, 812b, 912b, 1012b, 1112b, 1212b, 1312b, 1412b, 1512b, 112c, 1212c, 1312c, 1412c, 1512c may be coated with a surface coating or roughened to promote boney growth onto the blades 112b, 212b, 312b, 412b, 512b, 612b, 712b, 812b, 912b, 1012b, 1112b, 1212b, 1312b, 1412b, 1512b, 112c, 2 312c, 412c, 512c, 612c, 712c, 812c, 912c, 1012c, 1112c, 1212c, 1312c, 1412c, 1512c and onto the interbody/index implant 110, 210, 310, 410, 510, 610, 710, 810, 910, 1010, 1110, 1210, 1310, 1410, 1510. The blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be constructed of the same or different material as the interbody device or spacer 110, 210, 310, 410, 510, 610, 710, 810, 910, 1010, 1110, 1210, 1310, 1410, 1510.

The blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be solid or open architecture to further allow for bone incorporation and with or without a lattice/mesh work. The blades are constructed and arranged to pierce the cortices between the sacrum and ilium for transfixing the joint.

The blades 112b, 212b, 312b, 412b, 512b, 612b, 712b, 812b, 912b, 1012b, 1112b, 1212b, 1312b, 1412b, 1512b, 112c, 1212c, 1312c, 1412c, 1512c may be stacked as dual or multiple so that a space lies between them thereby catching bone between the blades when engaged or in the mounted configuration.

During insertion the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be designed to be placed in a non-deployed position or in the insertion configuration.

The deployed blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c can be designed to extend past the perimeter or the peripheral walls 114, 214, 314, 414, 514, 614, 714, 814, 914, 1014, 1114, 1214, 1314, 1414, 1514 of the implant 110, 210, 310, 410, 510, 610, 710, 810, 910, 1010, 1110, 1210, 1310, 1410, 1510 for any measurement and any angle.

A space left in the interbody device or interbody spacer 110, 210, 310, 410, 510, 610, 710, 810, 910, 1010, 1110, 1210, 1310, 1410, 1510 when the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c are deployed in the expanded configuration will allow bone integration to that now empty space within the peripheral wall 114, 214, 314, 414, 514, 614, 714, 814, 914, 1014, 1114, 1214, 1314, 1414, 1514. The deployed blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be designed to rotate from anywhere between zero and three hundred sixty degrees (0-360°) and locked at any angle relative to the peripheral wall 114, 214, 314, 414, 514, 614, 714, 814, 914, 1014, 1114, 1214, 1314, 1414, 1514.

Deployment of the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may utilize a screw/rotational like mechanism, such as the shaft 112a, 212a, 312a, 412a, 512a, 612a, 712a, 812a, 912a, 1012a, 1112a, 1212a, 1312a, 1412a and 1512a which can decrease the forces applied parallel to the front and back of the interbody spacer 110, 210, 310, 410, 510, 610, 710, 810, 910, 1010, 1110, 1210, 1310, 1410, 1510 that are common with traditional locking devices and interbody spacers. These front to back forces may be undesirable because the patient's spinal cord is typically positioned behind the spacer 110, 210, 310, 410, 510, 610, 710, 810, 910, 1010, 1110, 1210, 1310, 1410, 1510 in the mounted configuration.

The implant, interbody device or interbody spacer 110, 210, 310, 410, 510, 610, 710, 810, 910, 1010, 1110, 1210, 1310, 1410, 1510 preferably includes a lattice or web inside the perimeter of the peripheral walls 114, 214, 314, 414, 514, 614, 714, 814, 914, 1014, 1114, 1214, 1314, 1414, 1514 that may have a pore size that supports osseointegration as well matching the elastic properties of the bone. The lattice or web is preferably positioned to accommodate the shaft 112a, 212a, 312a, 412a, 512a, 612a, 712a, 812a, 912a, 1012a, 1112a, 1212a, 1312a, 1412a, 1512a and rotation of the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c. The lattice or web includes a relief opening or channel that accommodates the movement of the shaft 112a, 212a, 312a, 412a, 512a, 612a, 712a, 812a, 912a, 1012a, 1112a, 1212a, 1312a, 1412a, 1512a and rotation of the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c between the insertion and expanded configurations. The lattice or web is preferably designed, configured and positioned within the peripheral walls 114, 214, 314, 414, 514, 614, 714, 814, 914, 1014, 1114, 1214, 1314, 1414, 1514 such that the shaft 112a, 212a, 312a, 412a, 512a, 612a, 712a, 812a, 912a, 1012a, 1112a, 1212a, 1312a, 1412a, 1512a and the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be inserted into a slot or aperture 114a, 214a, 314a, 414a, 514a, 714a, 914a, 614a, 618a, 1014a, 1114a, 1214a, 1314a, 1414a, 1514a after the peripheral wall 114, 214, 314, 414, 514, 614, 714, 814, 914, 1014, 1114, 1214, 1314, 1414, 1514 with the lattice or web are inserted between the bones V1, V2. Once the peripheral wall 114, 214, 314, 414, 514, 614, 714, 814, 914, 1014, 1114, 1214, 1314, 1414, 1514 with the lattice or web are inserted between the bones V1, V2, the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c and/or the shaft 112a, 212a, 312a, 412a, 512a, 612a, 712a, 812a, 912a, 1012a, 1112a, 1212a, 1312a, 1412a, 1512a are inserted through the slot 114a, 214a, 314a, 414a, 514a, 714a, 914a, 614a, 618a, 1014a, 1114a, 1214a, 1314a, 1414a, 1514a until located in their final position and the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c and/or the shaft 112a, 212a, 312a, 412a, 512a, 612a, 712a, 812a, 912a, 1012a, 1112a, 1212a, 1312a, 1412a, 1512a may be locked into their position and the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c rotated through the relief space or channel to engage the bones V1, V2. The blades 112b, 212b, 312b, 412b, 512b, 612b, 712b,

812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be comprised of any number of biocompatible materials that are designed or configured to withstand the normal operating conditions, take on the general shape and size and perform the desired functions of the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1112c, 1212c, 1012c, 1312c, 1412c, 1512c.

The rotation of the shaft 112a, 212a, 312a, 412a, 512a, 612a, 712a, 812a, 912a, 1012a, 1112a, 1212a, 1312a, 1412a, 1512a is preferably utilized to extend and retract the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c to and from the insertion and expanded configurations.

During use, the rotation of the shaft 112a, 212a, 312a, 412a, 512a, 612a, 712a, 812a, 912a, 1012a, 1112a, 1212a, 1312a, 1412a, 1512a is relatively accessible to the surgeon to deploy and retract the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c.

The blades 112b, 212b, 312b, 412b, 512b, 612b, 712b,

812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c can designed for ninety degree (90°) interface with the adjacent bone thereby minimizing the skiving of other stand-alone locking devices. If used in the ninety degree (90°) option optimal controlled compression across the device 110, 210, 310, 410, 510, 610, 710, 810, 910, 1010, 1110, 1210, 1310, 1410, 1510 can further the ability of getting a fusion between the two adjacent bones or bone fragments, such as the adjacent vertebral bodies V1, V2. Safety advantages of the preferred interbody spacer 110, 210, 310, 410, 510, 610, 710, 810, 910, 1010, 1110, 1210, 1310, 1410, 1510 include the ability to recess the interbody spacer or device 110, 210, 310, 410, 510, 610, 710, 810, 910, 1010, 1110, 1210, 1310, 1410, 1510 and still easily deploy the locking mechanism without obstruction from existing bone, new grafts or other implants. The safety advantages include deploying blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c without pushing the interbody spacer 110, 210, 310, 410, 510, 610, 710, 810, 910, 1010, 1110, 1210, 1310, 1410, 1510 back toward the patient's spinal cord. Further advantages include ability to seat the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c further back, thereby increasing the ability to lock into solid bone and not bone spurs. Safety advantages also include the ability to seat the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c further back on the vertebral bodies V1, V2, thereby decreasing, if not eliminating, the complications seen with shearing off small pieces of bone that may occur with anterior locking devices. In addition, safety advantages and stability may be added with the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c (open architecture/fence architecture) actively participating with the possible fusion goals.

The ability to individually place blades/axes 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c with independent engagement technology allows the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c to be used with the expanding market of expandable interbody devices. In these expandable non-static devices, the blades/axes 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be incorporated into either side, thereby not affecting the expansion component of the implant or interbody spacer 110, 210, 310, 410, 510, 610, 710, 810, 910, 1010, 1110, 1210, 1310, 1410, 1510.

The pivotable blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be used with devices that are put in from various positions since the direct impact of other devices is not needed because the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c are deployed and/or retracted using the turning mechanism or rotation of the shaft 112a, 212a, 312a, 412a, 512a, 612a, 712a, 812a, 912a, 1012a, 1112a, 1212a, 1312a, 1412a, 1512a.

The implant or interbody spacers 110, 210, 310, 410, 510, 610, 710, 810, 910, 1010, 1110, 1210, 1310, 1410, 1510 may be implanted and then angled into a final position or mounted configuration and the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c can still be used to lock the interbody spacer 110, 210, 310, 410, 510, 610, 710, 810, 910, 1010, 1110, 1210, 1310, 1410, 1510 in the mounted configuration. As a non-limiting example, a banana cage may be pushed anteriorly after initial insertion, which does not allow for directly hitting in screws/nails to secure, but the ax fastener 112, 212, 312, 412, 512, 612, 712, 812, 912, 1012, 1112 could use an articulating screw driver to turn the shaft 112a, 212a, 312a, 412a, 512a, 612a, 712a, 812a, 912a, 1012a, 1112a, 1212a, 1312a, 1412a, 1512a and deploy the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c.

In the preferred embodiments, an inserter 16 may be utilized to insert the interbody spacer 110, 210, 310, 410, 510, 610, 710, 810, 910, 1010, 1110, 1210, 1310, 1410, 1510 and deploy the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c. The inserter 16 may be removably attached to the locking mechanism/shaft for the interbody spacer 110, 210, 310, 410, 510, 610, 710, 810, 910, 1010, 1110, 1210, 1310, 1410, 1510.

The inserter 16 may have a handle/grip mechanism to act as a counter torque as the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c are being rotated into the expanded configuration.

Unlike existing nails/screws/blades which are pushed into place using a sharp leading tip/edge, the ax fastener 112, 212, 312, 412, 512, 612, 712, 812, 912, 1012, 1112, 1212, 1312, 1412, 1512 provide sharp cutting edges along all/part of the perimeter to allow the engagement by rotating the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c into desired position.

The shaft 112a, 212a, 312a, 412a, 512a, 612a, 712a,

812a, 912a, 1012a, 1112a, 1212a, 1312a, 1412a, 1512a as described herein, may be comprised of any mechanism used to rotate the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c from the insertion configuration to the expanded configuration. Alternatively, the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may be rotated directly without the shaft 112a, 212a, 312a, 412a, 512a, 612a, 712a, 812a, 912a, 1012a, 1112a, 1212a, 1312a, 1412a, 1512a. The blades 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c may have a “cutting” edge around the entire perimeter to assist in placement and engagement of the blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c with the adjacent bones.

The preferred interbody spacers 110, 210, 310, 410, 510, 610, 710, 810, 910, 1010, 1110, 1210, 1310, 1410, 1510 may be comprised of any interbody device including disc replacement devices. The ax fastener 112, 212, 312, 412, 512, 612, 712, 812, 912, 1012, 1112 may be utilized with any orthopedic device for anchoring the orthopaedic implant, including anchoring to long bones and/or bone fragments. The ax fastener 112, 212, 312, 412, 512, 612, 712, 812, 912, 1012, 1112 may be utilized for sacroiliac (“SI”) joint fixation devices, flat bone fixation, including cranium, assisting to lock/secure components of joint replacement devices, with locking devices that rotates single, dual, independent and tandem axe blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c to secure to bones, blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that allow incorporation of graft/bone through or interdigitate with the locking securing mechanism, blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c for rotating locking/securing mechanism that can be place within or on perimeter of the device, blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that come independent to the primary implant and can be attached to the primary implant before implantation of the primary implant, blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that may come as part of the “whole” primary implant as one unit, blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that may come independent of the primary implant and attach prior to placement of the primary implant or after insertion of the primary implant and/or independent blades 112b, 212b, 312b, 412b, 512b, 612b, 712b, 812b, 912b, 1012b, 1112b, 1212b, 1312b, 1412b, 1512b, 112c, 212c, 312c, 412c, 512c, 612c, 712c, 812c, 912c, 1012c, 1112c, 1212c, 1312c, 1412c, 1512c that may be attached to the primary implant as part of single “whole” unit, prior to insertion as free standing devices, or after insertion as free standing devices in with multiple arrangements.

The interbody spacer allows for transfixing a joint. In the embodiments, the peripheral wall member has two end walls spaced apart by adjoining side walls defining an internal space therebetween. Additional side walls forming a cage member. The peripheral wall having a centrally disposed aperture formed in the side walls for receipt of a shaft extending between and rotationally coupled to the apertures. At least one blade is coupled to the shaft, the blade is constructed and arranged to pierce the cortices of adjoining vertebral bodies upon rotation of the shaft. The blade is rotated from an insertion configuration to an expanded configuration to lock said blade to adjoining vertebral bodies. The peripheral wall includes opposing first and second side walls to form a cage, the cage having openings to allow the blade to extend beyond the upper and lower planes of said peripheral wall. In one embodiment, the interbody spacer is constructed and arranged to pierce the lock the sacrum and ilium for transfixing the joint.

It will be appreciated by those skilled in the art that changes could be made to the embodiments described above without departing from the broad inventive concept thereof. It is understood, therefore, that this invention is not limited to the embodiments disclosed, but it is intended to cover modifications within the spirit and scope of the present invention as defined by the present description.

1. An interbody spacer for transfixing a joint comprising: a peripheral wall member having two end walls spaced apart by adjoining side walls defining an internal space therebetween; a centrally disposed aperture formed in said side walls for receipt of a shaft extending between and rotationally coupled to said apertures; and at least one blade coupled to said shaft, said blade constructed and arranged to pierce the cortices of adjoining vertebral bodies upon rotation of said shaft; wherein said blade is rotated from an insertion configuration to an expanded configuration to lock said blade to adjoining vertebral bodies.

2. The interbody spacer according to claim 1 wherein said blade is positioned between upper and lower planes of said peripheral wall forming an insertion configuration, said blade extends beyond the upper and lower planes of said peripheral wall in an expanded configuration.

3. The interbody spacer according to claim 1 wherein each said aperture is a slot, said shaft slidably coupled to each said slot.

4. The interbody spacer according to claim 1 including a lattice formed within said internal space of said peripheral wall, said lattice including a relief space for receipt of each said blade in the insertion configuration.

5. The interbody spacer according to claim 1 wherein each said blade is constructed and arranged to obtain a ninety degree (90°) interface with adjacent bone.

6. The interbody spacer according to claim 1 wherein each said blade includes a cutting edge formed around the perimeter of the blade and a surface of each said blade is treated to promote boney growth.

7. The interbody spacer according to claim 1 including a second shaft and blade.

8. The interbody spacer according to claim 7 wherein said second shaft is offset from the first shaft.

9. The interbody spacer according to claim 1 wherein said blade is constructed and arranged to lock the sacrum and ilium for transfixing the joint.

10. The interbody spacer according to claim 1 wherein said peripheral wall includes opposing first and second side walls to form a cage, said cage having openings to allow said blade to extend beyond the upper and lower planes of said peripheral wall.

11. A bone spacer for connecting a first bone portion to a second bone portion, the bone spacer comprising:
a peripheral wall defining an internal space, the internal space including a lattice with a channel therein; a rotatable fastener connected to the peripheral wall, the rotatable fastener including a shaft, a first blade and a second blade, the rotatable fastener movable from an insertion configuration to an expanded configuration, the first and second blades positioned between upper and lower planes defined by the peripheral wall in the insertion configuration and extending beyond the upper and lower planes in the expanded configuration to engage the first and second bone portions.
